(19) United States
(12) Patent Application Publication
(10) Pub. No.: US 2021/0311049 A1
(43) Pub. Date: Oct. 7, 2021

(54) RAPID DIAGNOSTICS OF MYCOBACTERIA WITH LECTIN CONJUGATED PARTICLES

(71) Applicants: Hapuarachchige Surangi JAYAWARDENA, Huntsville, AL (US); Kavini Madushika RATHNAYAKE, Huntsville, AL (US); Unnati PATEL, Huntsville, AL (US); Andrew SENTELL, Huntsville, AL (US); James JOHNSON, Huntsville, AL (US); Melinda MUSTAIN, Huntsville, AL (US); Veer Venkata Naga Manohar DEVARASETTY, Huntsville, AL (US)

(72) Inventors: Hapuarachchige Surangi JAYAWARDENA, Huntsville, AL (US); Kavini Madushika RATHNAYAKE, Huntsville, AL (US); Unnati PATEL, Huntsville, AL (US); Andrew SENTELL, Huntsville, AL (US); James JOHNSON, Huntsville, AL (US); Melinda MUSTAIN, Huntsville, AL (US); Veer Venkata Naga Manohar DEVARASETTY, Huntsville, AL (US)

(21) Appl. No.: 17/268,500

(22) PCT Filed: Aug. 15, 2019
(86) PCT No.: PCT/US2019/046719

§ 371 Date: Feb. 15, 2021

Related U.S. Application Data
(60) Provisional application No. 62/764,728, filed on Aug. 15, 2018.

(51) Int. Cl. G01N 33/569

(52) U.S. Cl. CPC G01N 33/5695; G01N 2333/35; G01N 2446/80; G01N 2405/00; G01N 2400/00

(57) ABSTRACT

The present disclosure is generally directed to methods and systems for the rapid detection of mycobacteria in samples using lectin-conjugated silica coated magnetic nanoparticles (SMNPs). In this work, carbohydrates on the cell wall of the mycobacteria serve as binding sites for lectins conjugated on the surface of lectin-conjugated SMNPs. As the target species of mycobacteria bind to lectin-conjugated SMNPs, a precipitate forms, which can be magnetically separated from the bulk test solution to visually indicate the presence of the target species of mycobacteria. The present disclosure is utilized as an inexpensive and rapid point of care system in one embodiment. In another embodiment, the methods and systems are integrated into a lateral flow assay for rapid detection of the target species of mycobacteria. In yet another embodiment, the methods and systems are utilized to create a microfluidic detection device with increased sensitivity to mycobacteria in a sample.

### CROSS REFERENCE TO RELATED APPLICATIONS

This application claims priority to U.S. Provisional Application No. 62/764,728, entitled “Rapid Diagnostics of Mycobacteria with Lectin Conjugated Magnetic Nanoparticles” and filed on Aug. 15, 2018, which is incorporated herein by reference.

### RELATED ART

Tuberculosis is an infectious disease generally caused by Mycobacterium tuberculosis that exists as a latent and an active condition. Latent tuberculosis occurs when tuberculosis bacteria infect the body but remain in an inactive state. According to the World Health Organization Global Tuberculosis Report for 2018 (https://www.who.int/tb/publications/global_report/en/), approximately one quarter of the world's population, most commonly in Africa, Asia, and Latin America, are believed to be infected with tuberculosis. Though latent tuberculosis is not contagious, it can become active at a later time, leading to both negative health outcomes for the infected and to the spread of the disease. Active tuberculosis is contagious and marked by symptoms including prolonged coughing with or without blood, fever, night sweats, chills, fatigue, and unintentional weight loss. Populations with HIV are more likely to become infected with tuberculosis due to the suppression of their immune systems, and are also more likely to have latent tuberculosis progress to active tuberculosis. Tuberculosis results in over a million annual fatalities, with the majority of deaths occurring in developing countries.

Effective testing for tuberculosis allows for early treatment and potential prevention of disease spreading. Those diagnosed with latent tuberculosis infections may elect to take medications to reduce the risk of tuberculosis becoming active. Diagnosis of active tuberculosis may result in actions to prevent the spread of tuberculosis, such as covering the mouth when coughing, wearing a mask, and reducing contact with others while undergoing treatment, which generally involves the administration of antibiotics.

Common detection methods include the tuberculin skin test, nucleic acid amplification tests, culture methods, and conventional microscopy. These methods, however, are often time consuming (taking from hours to months), require expensive laboratory equipment, and must be conducted by a trained clinical technician. Additionally, the tuberculin skin test may result in false positives for those who have previously received a Bacille Calmette-Guerin vaccine to reduce the risk of severe tuberculosis. A point of care test for the presence of Mycobacterium tuberculosis should therefore be relatively inexpensive to manufacture, provide rapid and reliable results, and operate without requiring experienced clinical personnel, additional equipment, or instruments.

### SUMMARY

The present disclosure generally pertains to methods and systems for detecting target species of mycobacteria using lectin-conjugated particles. For example, lectin-conjugated nanoparticles and lectin-conjugated silica coated magnetic nanoparticles (SMNPs) are presented for the rapid detection of Mycobacterium tuberculosis and subsequent diagnosis and treatment of tuberculosis. In some embodiments, a system detects Mycobacterium smegmatis, a mimic of Mycobacterium tuberculosis that is often utilized in the study of tuberculosis due to its faster doubling time and lower biosafety level facility requirements. In addition to sharing many homologous genes, Mycobacterium smegmatis has the same cell wall structure as Mycobacterium tuberculosis, which is not conducive to Gram staining. Lectin-conjugated SMNPs can be used to bind carbohydrate epitopes in the cell wall of the target species of mycobacteria. Mycobacteria-bound particles precipitate in samples containing the target species of mycobacteria, such that magnetically separated precipitate indicates the presence of the mycobacteria. The disclosed methods may be conducted in less than five minutes and, when used with samples containing Mycobacterium tuberculosis, may result in the diagnosis of tuberculosis and the offer of subsequent treatments.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 is a schematic depicting lectin-conjugated silica coated magnetic nanoparticles (SMNPs) incubated with a mycobacteria-containing sputum sample to produce a precipitate that indicates the presence of the mycobacteria.

FIG. 2 is a schematic depicting the fabrication of the lectin-conjugated SMNPs of FIG. 1.

FIG. 3 is a flow chart illustrating an exemplary method for rapid detection of mycobacteria as shown in FIG. 1

FIGS. 4A-B depict an embodiment of an exemplary device for the rapid detection of mycobacteria, using the materials and process shown in FIG. 1.

FIG. 5 depicts another embodiment of an exemplary device for the rapid detection of mycobacteria, using the materials shown in FIG. 1 within a microfluidic system.

### DETAILED DESCRIPTION

The present disclosure is generally directed to methods and systems for the rapid detection of mycobacteria in samples using lectin-conjugated particles. More particularly, the methods and systems of the present disclosure may be used for the diagnosis and treatment of tuberculosis through the detection of Mycobacterium tuberculosis in sputum samples. In this regard, glycolipids or carbohydrate epitopes within the cell wall of the mycobacteria, such as carbohydrate epitopes of lipoarabinomannan (LAM) in the instance where Mycobacterium tuberculosis is probed, serve as a binding site for lectins conjugated on the surface of lectin-conjugated silica coated magnetic nanoparticles (SMNPs). As the target species of mycobacteria bind to lectin-conjugated SMNPs, a precipitate forms, which can be magnetically separated from the bulk test solution to visually indicate the presence of the target species of mycobacteria. The present methods and systems are utilized as an inexpensive and rapid point of care system in the field in one embodiment. In another embodiment, the methods and systems are integrated into a lateral flow assay for rapid detection of the target species of mycobacteria. In yet another embodiment, the methods and systems are utilized to create a microfluidic detection device with increased sensitivity to mycobacteria in a sample. In some embodiments, lectins are conjugated to magnetic particles, while in other embodiments, lectins are conjugated to non-magnetic particles.

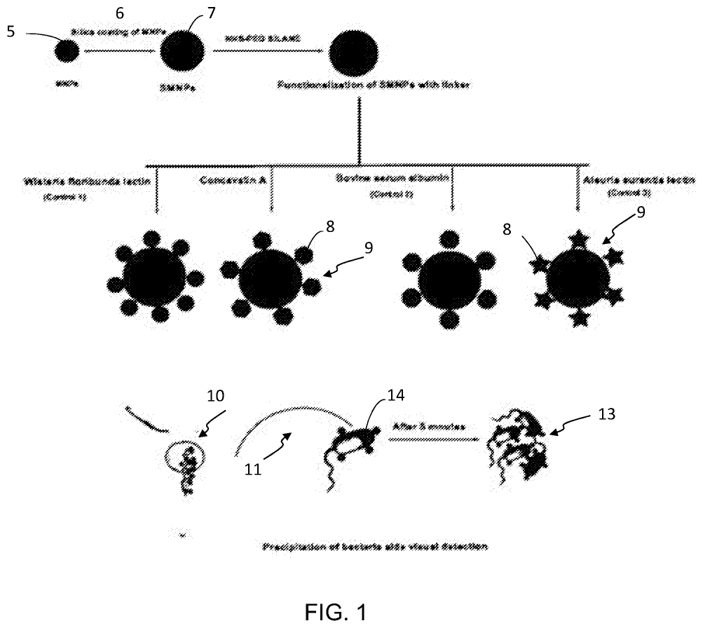

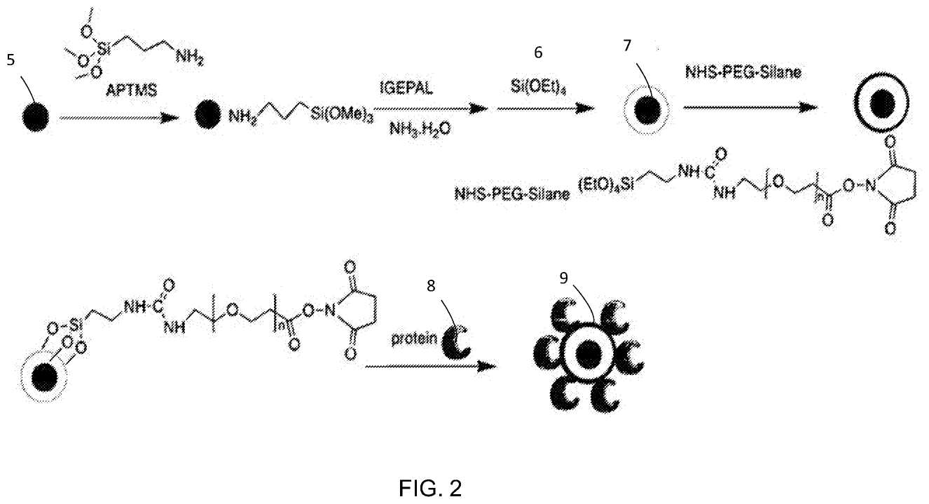

Referring to FIG. 1, materials and processes for the rapid detection of mycobacteria 14 and diagnosis of conditions associated with the mycobacteria 14 are shown. For the species of mycobacteria 14 of interest, a specific lectin or lectins 8 are chosen which bind to carbohydrates epitopes or glycolipids unique to or in abundance specifically on the cell wall of that species of mycobacteria 14. The lectins 8 are conjugated on the outer surface of SMNPs 7, as shown in detail in FIG. 2.

Referring to FIG. 2, magnetic nanoparticles 5 are first chemically functionalized and coated with silica 6. The magnetic nanoparticles 5 may include superparamagnetic iron oxides (SPIONs), such as Fe₂O₃ and Fe₃O₄, although other suitable magnetic materials are contemplated for use in the present disclosure. Magnetic nanoparticles 5 may be acquired from commercial vendors or fabricated according to a method similar to that disclosed in Sun et al. (S. Sun, H. Zeng, D. B. Robinson, S. Raoux, P. M. Rice, S. X. Wang and G. Li, J. Am. Chem. Soc., 2003, 126, 273-279.), which is hereby incorporated by reference in its entirety: iron (III) acetylacetonate (0.706 g, 2.0 mmol) is stirred with 1, 2-hexadecanediol (2.584 g, 10.0 mmol), oleylamine (2.820 mL, 6 mmol), and oleic acid (2.239 mL, 6.0 mmol) in benzyl ether (20 mL) and refluxed under N₂. The mixture is then heated to about 200° C. for approximately 2 hours followed by heating to about 280° C. for approximately another hour. After stirring at 400 rpm overnight, the resulting magnetic nanoparticle 5 solution is purified by adding ethanol (200-proof, 40 mL) and the mixture is centrifuged at 7000 rpm for about 10 minutes. A black precipitate is re-dispersed in hexane (30 mL) containing oleic acid (0.05 mL) and oleylamine (0.05 mL), and the mixture is then centrifuged at 6000 rpm for about 10 minutes. The precipitate is discarded, the supernatant is collected, and ethanol is added. After centrifugation, the precipitate is re-dispersed in hexane, completing the synthesis of magnetic nanoparticles 5.

A number of magnetic nanoparticles 5 are further stabilized and made biocompatible by incorporating a polymer matrix or producing a silica 6 coating. The number of magnetic particles 5 to incorporate per silica-coated particle may be varied as suitable for each application, as variation of the concentration of magnetic nanoparticles 5 affects the size of the resulting SMNPs 7. This silica 6 coating may be accomplished by any suitable method, including a method similar to the reverse micelle method of Selvan et al., (S T. Selvan, T. T. Tan and J. Y. Ying, Adv. Mater., 2005, 17, 1620-1625.), which is hereby incorporated by reference in its entirety. In an exemplary silica 6 coating method, magnetic nanoparticles 5 (0.5 mg) are dispersed in cyclohexane (22 mL) and a solution of APS in cyclohexane (60 μL, 0.1 mM) is added, followed by stirring the mixture at approximately room temperature for about 30 minutes. Next, IGEPAL (0.225 g) in cyclohexane (5 mL), NH₄OH (25%, 100 μL) and TEOS (100 μL), are added sequentially and the mixture is stirred for about 30 minutes, about 1 hour, and overnight, respectively, after the addition of each reagent. The product is purified by centrifugation at 15000 rpm for about 10 minutes and by re-dispersion in ethanol about three times to produce SMNPs 7. Variation of concentrations of IGEPAL may be undertaken to alter the size of resulting SMNPs 7, as suitable per application.

The SMNPs 7 may also be functionalized with a linker molecule, such as NHS-PEG-silanes, so that they may react and become conjugated to lectin 8. NHS-PEG-silane may range from about 1 kDa to about 5 kDa, or may be of any molecular weight such that the linker effectively conjugates lectin 8 to SMNPs 7 and retains the function of lectin 8 after conjugation. The amount of NHS-PEG-silane to use during functionalization is calculated based on size of SMNPs 7 after silica 6 coating and concentration of SMNPs 7. For instance, 1 mg/mL SMNPs 7 may be mixed with NHS-PEG-silane and stirred overnight. The solution may be purified by centrifugation at 15000 rpm for about 10 minutes, followed by re-dispersion in ethanol. Additional linker molecules capable of binding both the SMNPs 7 and lectin 8 and providing effective spacing of conjugated lectin 8 from SMNPs 7, such that lectin 8 may bind target carbohydrates, are contemplated for use in the present disclosure. Resulting SMNPs 7 range in diameter from about 200 nm to about 700 nm. This size range is selected to produce lectin-conjugated SMNPs 9 of about 400 nm to about 800 nm in diameter after lectin 8 conjugation.

Referring back to FIG. 1, lectin 8 may be Concanavalin A (Con A), Aleuria aurantia lectin (AAL), or any other suitable lectin 8 for the selective binding of the mycobacterial glycolipid or carbohydrate epitopes of choice. While point of care testing commonly employs antibody recognition of target antigens to produce a result, the use of lectins 8 to probe target antigens presents several advantages over antibody-based approaches. Lectins 8 are inexpensive to produce relative to the cost of antibodies, allowing more cost effective bulk production. Additionally, antibody-based products may require cold temperature storage or have reduced product life compared to lectin-based products, which may be comparably more stable and convenient to use in the field. Previous work in separations and detection has commonly employed antibodies as targeted recognition molecules, in part due to their strong affinities for their target antigens. While individual lectin-carbohydrate interactions are generally weaker than individual antibody-antigen interactions, the present work improves the binding strength of lectin-conjugated materials by leveraging multivalent interactions with target carbohydrate epitopes in the cell walls of mycobacteria 14. This multivalency, or ability to bind multiple antigens, is provided by conjugating multiple lectins 8 on each SMNP 7 and provides a greater overall affinity for carbohydrate epitopes on the cell wall of target species of mycobacteria 14 than an individual lectin-carbohydrate interaction would provide. Multivalent interactions of lectins 8 in the present work also serve to increase the rate of binding, allowing for rapid detection in times of less than about five minutes, or less than about one minute.

In the instance where the species of mycobacteria 14 is Mycobacterium tuberculosis or Mycobacterium smegmatis, a glycolipid that has particularly unique abundance is lipoarabinomannan. Con A has a high affinity for the mannan residues and AAL has a high affinity for the arabinose residues, making both Con A and AAL suitable choices for binding specifically to Mycobacterium tuberculosis or Mycobacterium smegmatis. When detection, diagnosis, or treatment of tuberculosis is a goal, assays for the presence of Mycobacterium tuberculosis are useful. However, development of assays is often undertaken using Mycobacterium smegmatis as a model due to its high similarities in genetic makeup and cell wall structure with Mycobacterium tuberculosis and its lower biosafety level requirements for facilities and fast growth.

Lectin 8 conjugation may be undertaken by the following exemplary process: N-hydroxysuccinimide (NHS) groups present on surface of linker-functionalized SMNPs 7 are used to conjugate the SMNPs 7 with amine groups of lectins 8. The lectins 8 are mixed with linker-functionalized SMNPs 7 and stirred overnight. The amount of lectin 8 required to conjugate 1 mg/mL of SMNPs 7 is calculated based on size and concentration of SMNPs 7. The solution is then centrifuged at about 15000 rpm for about 10 minutes and re-dispersed in water, producing lectin-conjugated SMNPs 9. Lectin 8 conjugation to SMNPs 7 can be analyzed using techniques such as dynamic light scattering, zeta potential measurements, and FTIR. Resulting lectin-conjugated SMNPs 9 generally range between about 400 nm and about 800 nm. When lectin-conjugated SMNPs 9 are less than about 400 nm in diameter, precipitate 13 may not form or may form in low amounts such that visualization without use of specialized equipment is not possible. When lectin-conjugated SMNPs 9 are greater than about 800 nm in diameter, they may self-precipitate, rendering detection methods and devices ineffective. Another parameter to consider in conjugating lectin 8 to SMNPs 7 is the lectin density. In one instance, 50 μg lectin 8 is present on 1 mg SMNPs 7. Increasing lectin density may increase binding interactions with carbohydrate epitopes, but may also result in reduced function of lectins 8 at some high densities due to packing. Reducing lectin density may decrease the number of binding events or increase times for precipitate 13 formation.

FIG. 1 displays a schematic for a study where Mycobacterium smegmatis is the species of mycobacteria 14 to be targeted. In this instance, Con A and AAL are lectins 8 that bind specifically to lipoarabinomannan within the cell wall of Mycobacterium smegmatis, while Wisteria floribunda lectin is a control lectin that has low binding affinity for lipoarabinomannan and bovine serum albumin is a protein not known to bind carbohydrates specifically. The Con A therefore binds to mannan residues of lipoarabinomannan and AAL binds arabinose residues, while Wisteria floribunda lectin and bovine serum albumin do not specifically bind lipoarabinomannan or other carbohydrates of the cell wall of Mycobacterium smegmatis. Con A and AAL are multivalent; they have multiple binding sites for their target carbohydrates or glycolipids. This multivalency allows Con A and AAL to bind multiple Mycobacterium smegmatis, having the effect of cross-linking and forming precipitate 13.

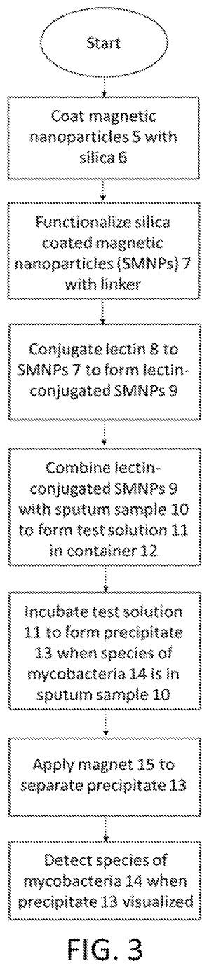

Referring to FIG. 3, after the formation of lectin-conjugated SMNPs 9, a test solution 11 is formed by combining lectin-conjugated SMNPs 9 with a sputum sample 10 from a human subject. Sputum is used because mycobacteria 14 are present in this fluid and it is relatively non-invasive to collect, allowing for its collection in the field where specialized, equipment, instrumentation, and personnel are not readily available. In the instance where Con A is lectin 8 conjugated to form lectin-conjugated SMNPs 9, approximately 1 mg of Con A-SMNPs can precipitate about 10⁶ CFU/mL of Mycobacterium smegmatis. Thus, adding about 1 mg of lectin-conjugated SMNPs 9 to about 10 μl of sputum sample 10 creates an effective test solution 11. In one instance, the concentration of Mycobacterium smegmatis in sputum sample 10 ranges from about 10⁵ CFU/mL to about 10⁸ CFU/mL at a volume of 10 μL. Concentrations below about 10⁵ CFU/mL Mycobacterium smegmatis may not form any precipitate 13 and concentrations above about 10⁸ CFU/mL may form nonspecific precipitate. The amount of lectin-conjugated SMNPs 9 in test solution 11 may be varied based on sputum sample 10 volume, mycobacterium affinity, mycobacterium concentration, detection method, target incubation time, and other such factors. For example, concentrations of less than about 5 mg/mL of lectin-conjugated SMNPs 9 may not be sufficient to produce visible precipitate 13 in test solution 11, while about 5 mg/mL to about 15 mg/mL of lectin-conjugated SMNPs 9 may quickly result in precipitation and is an exemplary range of concentrations for use in the present work. Concentrations of lectin-conjugated SMNPs 9 that are greater than about 15 mg/mL may result in unspecific aggregation. In some embodiments, a high final concentration of lectin 8 is desired in test solution 11 for rapid (less than one minute) incubation times and highly visible precipitate 13 formation. However, in other embodiments where incubation occurs in microfluidic channels where precipitate 13 size presents a potential for blockage, concentrations of lectin 8 in test solution 11 are reduced.

FIG. 3 illustrates that once test solution 11 is produced in a container 12, an incubation period results in the formation of precipitate 13 if the target species of mycobacteria 14 is in sputum sample 10. Incubation parameters may include time of incubation, test solution 11 temperature, agitation, and any other suitable factors to control the formation of precipitate 13. In embodiments where methods and systems of the present disclosure are undertaken in the field, container 12 may be a microcentrifuge tube or similar small, lidded vessel, such that test solution 11 components may be easily added. Incubation can be done by rolling sealed container 12 containing test solution 11 between the palms of the hands of an operator, such that precipitation occurs rapidly. For instance, incubation may occur in less than about five minutes, or in less than about one minute when large agglomerates 33 or precipitate 13 do not hinder the detection mechanism. In the embodiment where no instrumentation is used and container 12 is incubated by rolling it in the palm of an operator's hand, precipitate 13 formed when the target species of mycobacteria 14 is present in the test solution 11 is visible to the human eye. The hand-rolling provides heat and agitation to test solution 11 and facilitates rapid detection. A magnet 15 is applied to the exterior of container 12 to separate precipitate 13 and allow for better visualization. Magnet 15 may be a neodymium iron boron rare earth magnet or any other such magnet suitable for exerting magnetic forces on magnetic nanoparticles 5 The separation by magnet 15 is possible due to the presence of magnetic nanoparticles 5, which are not significantly visible to the human eye unless bound and cross-linked to mycobacteria 14, and in particular to Mycobacterium tuberculosis or Mycobacterium smegmatis. Precipitate 13 can be visualized by those who are colorblind, as it is not color-dependent.

After precipitate 13 is either determined to be present or not present in working solution 11, the presence or absence of the target species of mycobacteria 14 is determined as well. When no or substantially no precipitate 13 is formed after incubation, the result is that the species of mycobacteria 14 is not present in sputum sample 10 of the human subject, and the human subject may not be diagnosed with or treated for any connected condition or disease. For instance, when precipitate 13 indicates the presence of Mycobacterium tuberculosis, the human subject who provided sputum sample 10 may be diagnosed with tuberculosis or treated for tuberculosis. In this instance, treating may include the reduction of tuberculosis transmission or spreading from the human subject or the reduction of symptoms in the human subject. When no precipitate 13 or substantially no precipitate 13 is visible, the absence of Mycobacterium tuberculosis in sputum sample 10 is indicated, leading to no diagnosis of tuberculosis or treatment based on results from the present methods and systems.

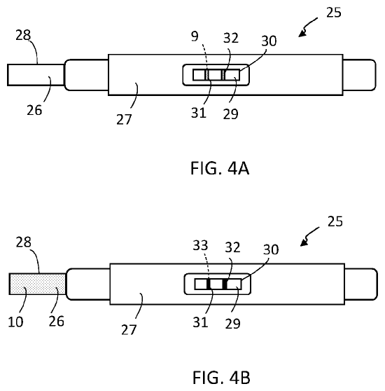

Referring to FIGS. 4A-B, one embodiment of the methods and systems for rapid detection of mycobacteria 14 using lectin-conjugated SMNPs 9 is through the use of a lateral flow assay 25 detection system. Lateral flow assay 25 is shown before application of sputum sample 10 in FIG. 4A and after sputum sample 10 has been applied in FIG. 4B. As shown in FIG. 4A-B, lateral flow assay 25 comprises a nitrocellulose membrane 26 surrounded by a plastic body 27. Nitrocellulose membrane 26 allows for the transport of sputum sample 10 from an application region 28 where it is applied, to a test region 29 where binding reactions occur between sputum sample 10 and embedded molecules or compounds in nitrocellulose membrane 26. Application region 28 is located at a first end of lateral flow assay 25, while test region 29 is located within plastic body 27 between said first end and a second end of lateral flow assay 25. Plastic body 27 protects test region 29 during handling and use, such that embedded molecules in two assay lines are not damaged or denatured and such that sample transport is undisturbed. A test window 30 within plastic body 27 is located above assay lines of test region 29 so that test outcomes may be visualized and results may be determined.

Assay lines within test region 29 may comprise two lines: a test line 31 and a control line 32. In embodiments not depicted, lateral flow assay 25 may include multiple test lines to detect various molecules or compounds. In the embodiment depicted in FIGS. 4A-B, test line 31, located beneath test window 30 closest to the first end of lateral flow assay 25, becomes visible when species of mycobacteria 14 is present in sputum sample 10. In FIG. 4A, test line 31 is a line of lectin-conjugated SMNPs 9 embedded in nitrocellulose membrane 26. Control line 32, located beneath test window 30 closest to the second end of lateral flow assay 25, becomes visible when lateral flow assay 25 has been operated correctly, regardless of the presence or absence of species of mycobacteria 14 in sputum sample 10. Lack of color along control line 32 after application of sputum sample 10 and a pre-determined incubation time indicates that lateral flow assay 25 was not operating correctly and that test line 31 results should be disregarded. Test line 31 may be located nearest the second end of lateral flow assay 25 and control line 32 may be located nearest the first end of lateral flow assay 25 in other embodiments of lateral flow assay 25 not depicted herein.

FIG. 4B displays lateral flow assay 25 after application of sputum sample 10 on application region 28. Sputum sample 10 moves to test region 29 by wicking along nitrocellulose membrane 26. Incubation time includes the time necessary for sputum sample 10 movement, as well as incubation along the assay lines. As sputum sample 10 reaches test line 31, any target species of mycobacteria 14 present in sputum sample 10 binds to lectins 8 of the lectin-conjugated SMNPs 9 and agglomerates 33 are formed along the line, leading to a shaded or colored line that is visible to the user. As sputum sample 10 reaches control line 32, it interacts with embedded molecules, which may be a variety of anti-human antibodies, and binds to form a colored or shaded line product. In embodiments where lectin 8 is Con A or AAL, the presence of agglomerates 33 at test line 31 indicates the presence of Mycobacterium tuberculosis in sputum sample 10 of a human subject. In this case, the human subject is diagnosed with tuberculosis and may begin treatment for the disease. When lectin 8 is Con A or AAL, the absence of agglomerates 33 at test line 31 indicates the absence of Mycobacterium tuberculosis in sputum sample 10 of the human subject, and the human subject is not diagnosed with tuberculosis.

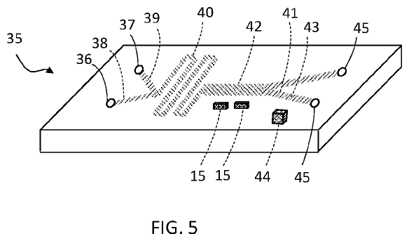

Referring to FIG. 5, another embodiment of the methods and systems of the present disclosure is schematically displayed. Microfluidic detection device 35 is shown with reagent inlet 36 and sample inlet 37 for conducting fluids and solutions into the device. Solutions may be applied by syringe injection, pipetting, or any other such suitable application technique. A solution containing lectin-conjugated particles, including lectin-conjugated SMNPs 9, may be applied to microfluidic detection device 35, such that it travels along at least one reagent channel 38 in the interior of the device. Similarly, a solution containing sputum sample 10 may be applied to microfluidic detection device 35, such that it travels along at least one sample channel 39 in the interior of the device. Channels are typically rectangular in cross section, but may be of any shape conducive to the unimpeded transport of solution components. Channel dimensions may range from about 10 μm to about 10 mm, though other dimensions are possible, and may be tailored to the size of solution components and potential aggregates. At least one reagent channel 38 and at least one sample channel 39 meet at a mixing region 40, where components of each channel are combined. Lectin-conjugated SMNPs 9 bind the target species of mycobacteria 14 when it is present in sputum sample 10, forming indicator particles 41. In the instance where the lectin 8 is Con A or AAL, bound mycobacteria 14 are Mycobacterium tuberculosis found in sputum sample 10 from a human subject.

Following mixing region 40, solutions enter separation region 42, where at least one magnet 15 is embedded in the device and configured to separate indicator particles 41 from unbound lectin-conjugated SMNPs 9 and unbound mycobacteria 14 or other solution components. In this regard, a magnetic field generated by at least one magnet 15 may apply a force (attract or repel) to the indicator particles due 41 thereby moving or separating them relative to the unbound lectin-conjugated SMNPs 9. In one embodiment, at least one magnet 15 separates indicator particles 41 using microfluidic magnetophoretic separations, which separate micro- or nano-scale magnetic particles within the microfluidic detection device 35. At least one magnet 15 may include situations where two or more magnets 15 are placed along a length of separation region 42, and magnets 15 may be of equal or unequal size, magnetic strength, or distance from separation region 42. As magnetic particles travel along separation region 42, they are initially attracted towards the first magnet 15. The movement of particles depends on parameters such as the strength and position of the magnets 15, the magnetic permeability of the particles, and the size of the particles. Thus, when particles have similar properties but different sizes, larger particles experience greater drag forces and reduced movement compared to smaller particles. Thus, larger particles may require more magnets 15 or higher magnetic strength to migrate the same distance as a smaller particle. In the present disclosure, indicator particles 41 are larger than unbound lectin-conjugated SMNPs 9, such that, while both will experience movement due to magnetic forces, the size difference between the particles will alter their movement and paths in separation region 42, resulting in separation of indicator particles 41. Non-magnetic particles and components of the solution will not be drawn into trajectories by the at least one magnet 15. Other magnetic or non-magnetic separation strategies may be implemented on microfluidic detection device 35, such that indicator particles 41 are separated from other solution components. When non-magnetic separation strategies are employed in microfluidic detection device 35, lectin-conjugated particles may be lectin-conjugated non-magnetic particles, including lectin-conjugated microparticles and lectin-conjugated nanoparticles.

Separated indicator particles 41 may then enter a detection region 43 comprising a built-in detector 44. This detector 44 may be a cell counting device, a digital counter, or any other detector capable of counting indicator particles 41 as they pass through detection region 43 and communicating the resulting number to an operator. When the resulting number of indicator particles 41 exceeds a predetermined threshold, the result indicates the presence of target species of mycobacteria 14 and any corresponding disease or condition. When the resulting number of indicator particles 41 is less than a predetermined threshold, the result indicates the lack or low concentration of target species of mycobacteria 14 and fails to indicate any corresponding disease or condition associated with its presence or high concentration. Predetermined thresholds may be determined empirically or may be obtained from published studies or materials. Solutions exit microfluidic detection device 35 through at least one outlet 45.

When Mycobacterium tuberculosis is present in sputum sample 10, indicator particles 41 are formed as Con A or AAL binds lipoarabinomannan in mixing region 40. Detection of the resulting indicator particles 41 at levels above a threshold indicate that the human subject who provided sputum sample 10 has tuberculosis and diagnosis of tuberculosis is indicated. Microfluidic detection device 35 may be more expensive to prepare and complicated to operate than other embodiments of the present methods and systems, but it also affords the highest sensitivity to low concentrations of Mycobacterium tuberculosis and other target species of mycobacteria 14. Thus, earlier detection of tuberculosis is facilitated by use of a device such as microfluidic detection device 35.

In some embodiments, lectins 8 may be conjugated to non-magnetic particles, including nanoparticles and microparticles, for use in the present methods and systems. Non-magnetic particles may include polymeric particles, liposomes, dendrimers, silica particles, carbon nanotubes and particles, DNA nanostructures, micelles, and any other non-magnetic nano- or micron-sized material capable of conjugation with multiple lectins 8. In these instances, lectins 8 are conjugated on the non-magnetic particles to form lectin-conjugated particles, such that lectins 8 provide multivalent interactions with their target glycolipid residues or carbohydrate epitopes. In non-magnetic systems, lectin-conjugated particles may form precipitates 13, agglomerates 33, or mycobacteria-particle complexes upon binding their target species of mycobacteria 14, and are thus separated from bulk unbound mycobacteria 14 and lectin-conjugated particles by their binding interactions. In some embodiments, further sorting of mycobacteria-nanoparticle complexes may be undertaken using size-based, affinity-based, or charge-based separation methods or any other suitable sorting or separation technique.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

Now, therefore, the following is claimed:

1. A method of detecting Mycobacterium tuberculosis comprising:
obtaining a sputum sample from a human subject;
combining the sputum sample with lectin-conjugated silica coated magnetic nanoparticles (SMNPs) to form a test solution within a container;
incubating the test solution, such that when Mycobacterium tuberculosis is present in the test solution, lectins of the lectin conjugated SMNPs bind a glycolipid of a cell wall of Mycobacterium tuberculosis to form a precipitate, and when Mycobacterium tuberculosis is not present in the test solution, substantially no precipitate is formed;
applying a magnet to separate the precipitate within the container, such that the separated precipitate is visible to the human eye; and
determining that the human subject carries Mycobacterium tuberculosis when the precipitate is visible.

2. The method of claim 1, wherein the incubating is performed by rolling the container of the test solution between palms of hands of an operator.

3. The method of claim 1, wherein the glycolipid is lipoarabinomannan and the lectin is Concanavalin A or Aleuria Aurantia Lectin (AAL).

4. The method of claim 1, wherein formation of precipitate occurs in less than about five minutes.

5. The method of claim 1, further comprising the step of diagnosing the human subject with tuberculosis when precipitate is visible.

6. The method of claim 5, further comprising the step of treating the human subject for tuberculosis, wherein the treating comprises reducing tuberculosis spreading or symptoms.

7. A tuberculosis diagnosis system comprising:
a test solution, comprising lectin-conjugated silica coated magnetic nanoparticles (SMNPs) and a sputum sample, wherein the lectin-conjugated SMNPs comprise magnetic nanoparticles coated with silica and lectins conjugated to a surface of the coated silica, and wherein the sputum sample is provided by a human subject;
a container for incubating the test solution, wherein incubation results in formation of a precipitate when Mycobacterium tuberculosis is present in the sputum sample and incubation results in substantially no precipitate when Mycobacterium tuberculosis is not present in the sputum sample; and
a magnet configured to separate precipitate when it is formed, such that visualization of precipitate indicates tuberculosis in the human subject.

8. The system of claim 7, wherein the lectins are Concanavalin A.

9. The system of claim 7, wherein the lectins are Aleuria Aurantia Lectin (AAL).

10. The system of claim 7, wherein the magnetic nanoparticles comprise superparamagnetic iron oxides.

11. The system of claim 7, wherein the lectin-conjugated SMNPs have diameters between about 400 nm and about 800 nm.

12. The system of claim 7, wherein the test solution comprises about 1 mg of lectin conjugated SMNPs and about 10 μl of sputum sample.

13. The system of claim 7, wherein the test solution is incubated for less than about five minutes.

14. A method of operating a tuberculosis detection device comprising:
obtaining a sputum sample from a human subject;
contacting the sputum sample with lectin-conjugated particles within the detection device, such that, when Mycobacterium tuberculosis is present in the sputum sample, lectins on the lectin-conjugated particles bind a glycolipid residue on a cell wall of Mycobacterium tuberculosis and form mycobacteria-particle complexes;
detecting the mycobacteria-particle complexes, wherein said mycobacteria-particle complexes are separated from unbound Mycobacterium tuberculosis and unbound lectin-conjugated particles by their binding interactions.

15. The method of claim 14, wherein the glycolipid residue is mannan and the lectin is Concanavalin A.

16. The method of claim 14, wherein the glycolipid residue is arabinose and the lectin is Aleuria Aurantia Lectin (AAL).

17. The method of claim 14, wherein the detecting step occurs less than about five minutes after contacting the sputum sample with lectin-conjugated particles.

18. The method of claim 14, further comprising the step of diagnosing the human subject with tuberculosis when a number of detected mycobacteria-particle complexes exceeds a threshold.
